(19) United States
(12) Plant Patent Application Publication
(10) Pub. No.: US 2006/0143758 P1
(43) Pub. Date: Jun. 29, 2006

(54) CHRYSANTHEMUM PLANT NAMED 'CORHELLAS'

(50) CHRYSANTHEMUM MORIFOLIUM; CHRYSANTHEMUM

(75) Inventor: Mark Roland Boeder, The Hague (NL)

Correspondence Address: STEPTOE & JOHNSON LLP, 1330 CONNECTICUT AVENUE, N.W., WASHINGTON, DC, 20036, US

(73) Assignee: Chrysanthemum Breeders Association N.V., Aalsmeer (NL)

(21) Appl. No.: 11/020,522

(22) Filed: Dec. 27, 2004

(51) Int. Cl. A01H 5/00

(52) U.S. Cl. PLT/290

(57) ABSTRACT

A chrysanthemum plant named ‘CORHELLAS’ characterized by its medium sized blooms with orange ray florets combined with a bronze center and prolific branching; natural season flower date September 20-27; blooming for a period of 5 weeks.

### BACKGROUND OF THE INVENTION

‘Corhellas’ is a product of a breeding and selection program for outdoor pot mums (garden mums) which had the objective of creating new chrysanthemum cultivars with a decorative type flower, a natural season flower date around September 20-27 (week 39); blooming for a period of 5 weeks. The new plant of the present invention comprises a new and distinct cultivar of Chrysanthemum plant. ‘Corhellas’ is a seedling resulting from a crossing program, which was set up by a previous breeder, and which records are unknown to the inventor. The new and distinct cultivar was discovered and selected as one flowering plant by Mark Roland Boeder on a cultivated field in Rijsenhout, The Netherlands in 2001. The first act of asexual production of ‘Corhellas’ was accomplished when vegetative cuttings were taken from the initial selection in 2001 in a controlled environment in Rijsenhout, The Netherlands, and propagated further at this location. The new cultivar has been found to retain its distinctive characteristics through successive propagations.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

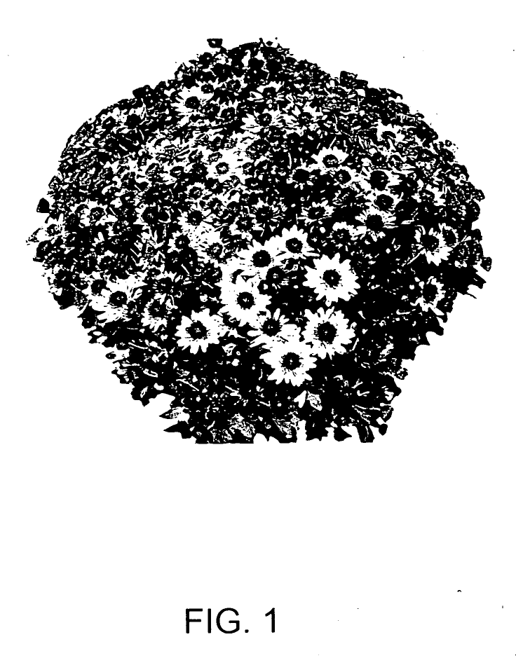

FIG. 2 shows the various stages of bloom of the new cultivar.

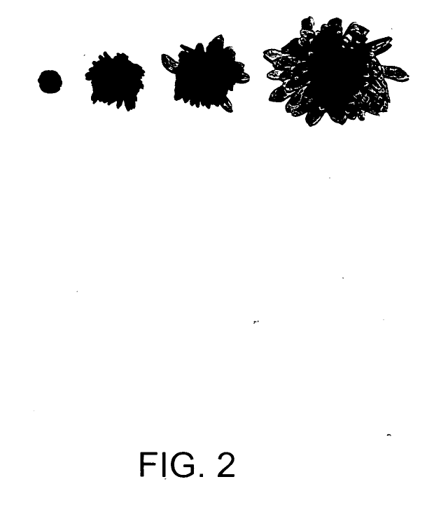

FIG. 3 shows the foliage of the new cultivar.

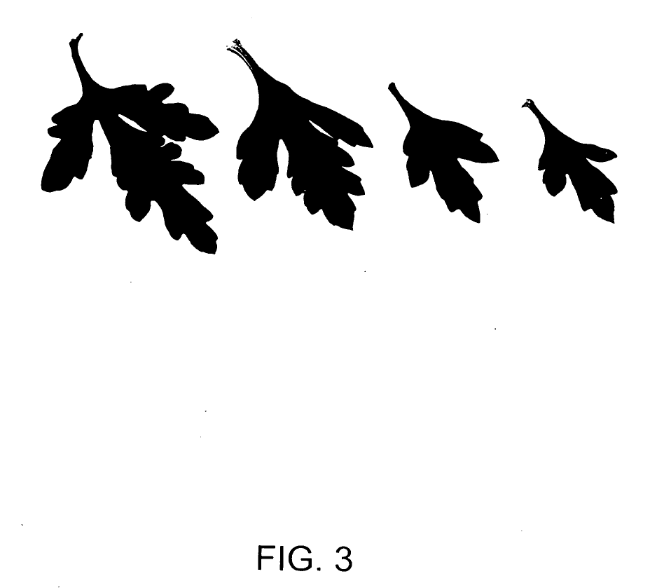

### DESCRIPTION OF THE INVENTION

This new variety of chrysanthemum is of the botanical classification Chrysanthemum morifolium L. The observations and measurements were gathered from plants grown out door in Rijsenhout, The Netherlands under natural day length and temperature and planted in week 24 in 2004. The natural blooming date of this crop was September 20-27 (week 39). The average height of the plants was 25 cms. No growth retardants were used. No tests were done on disease or insect resistance or susceptibility. No tests were done on cold or drought tolerance. This new variety produces medium sized blooms with orange ray florets and a bronze center blooming for a period of 5 weeks.

From the cultivars known to inventor the most similar existing cultivars in comparison to ‘Corhellas’ are ‘Iduna’ (U.S. Plant Pat. No. 14,925) and ‘Mars’ (U.S. Plant Pat. No. 14,678). When ‘Corhellas’, ‘Iduna’ and ‘Mars’ are being compared the following similarities and differences are noticed: All three varieties have medium sized two tone blooms with a dark center. The differences of ‘Corhellas’ and ‘Iduna’ and ‘Mars’ is (1) Natural flowering date. (1) When planted in week 24, ‘Iduna’ and ‘Mars’ flower at a later date than ‘Corhellas’.

The following is a description of the plant and characteristics that distinguish ‘CORHELLAS’ as a new and distinct variety. The color designations are taken from the plant itself. Accordingly, any discrepancies between the color designations and the colors depicted in the photographs are due to photographic tolerances. The color chart used in this description is: The Royal Horticultural Society Colour Chart, edition 1995.

TABLE 1
Botanical Description of CULTIVAR ‘CORHELLAS’

| | |
|---|---|
| Bud | |
| Size | Small; cross-section 0.5 cm, height 0.3 cm |
| Outside Color | Yellow-green 144D |
| Involucral bracts | 2 rows, length 7 mm, width 3 mm |
| Involucral bracts | Not present |
| among disc-florets | |
| Involucral bracts | Green 138C |
| color | |
| Bloom | |
| Type | Decorative |
| Height | 1 cm |
| Size | Medium |
| Fully Expanded | 4-5 cm |
| Peduncle length | 7 cm |
| Peduncle color | Green 143C-143D |
| Number of blooms | Approx. 7 blooms per branch |
| per branch | |
| Performance on | 5 weeks |
| the plant | |
| Seeds | Produced in small quantities, ovate grey-brown |
| | 199A, 1½ mm in length. |
| Fragrance | Typical chrysanthemum, slightly |
| Color | |
| Center of the flower | Immature Greyed-red 178A |
| | Mature Greyed-yellow 160A |
| Color of upper | Yellow-orange 21B to Greyed-orange 163C |
| surface of the ray- | |
| florets | |
| Color of the lower | Greyed-orange 166C |
| surface of the | |
| ray-florets | |
| Tonality from | A garden mum with orange flowers and a bronze |
| Distance | disc |
| Discoloration | Greyed-orange 163C |
| to color | |
| Ray florets | |
| Texture | Upper and under side smooth |
| Number | 160-180 |
| Cross-section | Flat |
| Longitudinal | Straight |
| axis of majority | |
| Length of | 0.4 cm |
| corolla tube | |
| Ray-floret margin | Entire |
| Ray-floret length | 2.2 cm |
| Ray-floret width | 0.4 cm |
| Ratio length/ | High |
| width | |
| Shape of tip | Dentate |
| Disc florets | |
| Disc diameter | 0.5 cm |
| Distribution | Few, only visible in mature stage |
| of disc florets | |
| Shape | Tubular |
| Color | Yellow-green 154D |
| Receptacle shape | Conical raised |
| Reproductive Organs | |
| Stamen | Present in disc florets only |
| Stamen color | Yellow-green 144A |
| Pollen | Present |
| Pollen color | Yellow 7A |
| Styles | Thick |
| Style color | Yellow 13A |
| Style Length | 0.4 cm |
| Stigma color | Yellow-green 144A |
| Stigma Width | 1 mm |
| Ovaries | Enclosed in calyx |
| Plant | |
| Form | Grown as a spray type potmum, outdoor |
| | mounded and round |
| Growth habit | Spherical shape |
| Growth rate | Medium |
| Height | 25 cm |
| Width | 45 cm |
| Stem Color | Greyed-brown 199C with at base Greyed-Red |
| | 182C |
| Stem Strength | Strong |
| Stem Brittleness | Not brittle |
| Stem Anthocyanin | Present at base of stem |
| Coloration | |
| Internode lenght | 2.5-3 cm |
| Length of | From top to bottom 17-19 cm |
| lateral branch | |
| Lateral branch | Green 138A-B |
| color | |
| Lateral branch, | Medium |
| attachment | |
| Branching (average | Good with 8-10 breaks after pinching |
| number of | |
| lateral branches) | |
| Natural season | September 20-27 |
| blooming date | |
| Foliage | |
| Leaf color | Upper side Green 139A-139B |
| | Under side Green 138A-138B |
| Color midvein | Upper side Green 139D |
| | Under side Green 138D |
| Size | Medium; length 6-9 cm, width 4-6 cm |
| Quantity (number | 13-15 |
| per lateral | |
| branch) | |
| Shape | Ovate |
| Texture upper side | Glabrous |
| Texture under side | Pubescent |
| Venation arrangement | Palmate |
| Shape of the margin | Serrated |
| Shape of Base of | Rounded |
| Sinus Between | |
| Lateral Lobes | |
| Margin of Sinus | Diverging |
| Between Lateral | |
| Lobes | |
| Shape of Base | Acute/obtuse |
| Apex | Mucronulate |
| Petiole lenght | 1-2 cm |
| Petiole color | Green 139D |

TABLE 2
Differences with the comparison varieties

| | ‘Corhellas’ | ‘Iduna’ | ‘Mars’ |
|---|---|---|---|
| Bloom size | 4-5 cm | 4.5-5 cm | 4.5-5 cm |
| Color center | Greyed-red | Greyed-red | Greyed-red |
| immature | 178A | 179A | 179A |
| bloom | | | |
| Color upper | Yellow-orange | Yellow-orange | Yellow-orange |
| side ray-florets | 21B to | 22C | 22B |
| | Greyed -orange | | |
| | 163C | | |
| Natural flowering | September | October | October |
| date (when planted | 20-27 | 4-11 | 11-18 |
| in week 24) | | | |

I claim:

1. A new and distinct variety of chrysanthemum plant as described and illustrated.
